(19) United States
(12) Patent Application Publication
(10) Pub. No.: US 2012/0203200 A1
(43) Pub. Date: Aug. 9, 2012

(54) DEVICE AND METHOD FOR DELIVERING MEDICINE INTO THE TYMPANIC CAVITY

(75) Inventors: George B. Kenney, Medfield, MA (US); Christian Pfeffer, Summerville, MA (US); Lev Rosenblum, Salt Lake City, UT (US)

(73) Assignee: ENTRATYMPANIC, LLC, Medfield, MA (US)

(21) Appl. No.: 13/501,073

(22) PCT Filed: Oct. 13, 2010
(86) PCT No.: PCT/US10/52569

§ 371 Date: Apr. 9, 2012

Related U.S. Application Data
(60) Provisional application No. 61/251,792, filed on Oct. 15, 2009.

(51) Int. Cl. A61M 5/00; A61M 5/32

(52) U.S. Cl. 604/506; 604/272; 604/60; 604/239

(57) ABSTRACT

A device for substance delivery to and/or extraction from the tympanic cavity, comprising: a housing, having a distal surface, a proximal surface that is opposite to the distal surface, and a circumscribed surface that connects the distal surface and the proximal surface; at least one piercing element having at least one proximal piercing end and at least one distal piercing end, disposed within the housing in such manner that the at least one distal piercing end is/are generally facing the distal surface of the housing; and a means for flexing the tympanic membrane toward the distal end of the at least one piercing element.

### CROSS-REFERENCE TO RELATED APPLICATIONS

The present application claims priority to PCT International Application No. PCT/US2010/052569, filed on Oct. 13, 2010, entitled “Device and Method for Delivering Medicine into the Tympanic Cavity” and to U.S. Provisional Patent Application No. 61/251,792, filed Oct. 15, 2009, entitled “Device and Method for Delivering Medicine into the Tympanic Cavity.” The entire content of the above-referenced applications is incorporated herein by reference.

### BACKGROUND OF THE INVENTION

1. The Field of the Invention

This invention relates, generally, to otologic devices and methods of treatments of various ear-related disorders, and more specifically to delivering medicine into the tympanic cavity.

2. Background and Relevant Art

The device and method in the present invention relate to delivery of medicine to the middle and/or inner ear and evacuation of fluid, if any, located in the tympanic cavity. The device and the method can be used, for example, for treatment and/or prevention of various ear-related ailments, such as acute otitis media.

It is frequently desirable to deliver various types of medicine into the tympanic cavity. Such medicine can be directed at treating ailments of the middle as well as the inner ear. For example, a medically effective amount of antibiotic and/or an anti-inflammatory drug(s) can be delivered through the tympanic membrane to treat middle ear infections. Currently, delivery of the drugs into the tympanic cavity is usually done when the tympanic membrane has ruptured or the patient has a previously-inserted tube in the membrane—i.e., non-surgical delivery of medicine to the tympanic cavity is usually done only when there is an existing perforation in the tympanic membrane, through which the medicine is delivered. The majority of the patients, however, do not have an existing perforation through which medicine can be delivered; consequently, such procedure is not available to them.

Alternatively, a health care provider can use a syringe to inject medicine through the tympanic membrane. However, this procedure can be dangerous for several reasons. First, the tympanic cavity houses a variety of vulnerable structures, such as the malleus, incus, stapes, facial nerve, and in some cases carotid artery. An accidental contact with any of these structures can result in adverse effects that range from pain and severe bleeding (in case of punctured carotid artery or branches of the internal jugular vein) to permanent disability, such as hearing loss.

Any incisions and/or perforations of the tympanic membrane in the posterosuperior and anterosuperior quadrants are highly discouraged because the most vulnerable structures located in the tympanic cavity are positioned proximately behind these two quadrants. Consequently, incisions and/or perforations of the tympanic membrane are usually performed in the posteroinferior and anteroinferior quadrants. Further, incisions and/or perforations made in the posteroinferior and anteroinferior quadrants must also be done with extreme care, and accidental penetration beyond minimal depth can cause severe injuries. Because the health care provider must insert the needle in a tiny area and with minimal penetration, the margin of error is very low, and incisions and/or penetrations of the tympanic membrane in children are usually performed under general anesthesia, to avoid accidental over-penetration or an unwanted penetration in a wrong location (e.g., a perforation in the posterosuperior quadrant or contact with the ear canal) as a result of the child's inability to remain stationary during the procedure.

This invention offers a novel way of safely delivering desired amounts of medicine into the tympanic cavity. The invention allows evacuation of fluid from the tympanic cavity, delivery of medicine into the tympanic cavity, and/or biopsy of the tympanic membrane by making a minute perforation in the membrane. Furthermore, the invention allows the delivery to be performed quickly, safely, without anesthesia, and without surgery by limiting the depth of penetration of and the location of penetration on the tympanic membrane, i.e., the device self-limits the depth of penetration as well as the location of the penetration. Also, the invention allows for safe removal of fluid accumulated in the tympanic cavity, as well as subsequent analysis of the fluid. Such analysis, for example, may entail a test for the presence of bacteria and a determination of the type of bacteria present. Consequently, the invention will reduce the need for systemic antibiotic treatment of ailments related to middle and inner ear in patients who do not have a perforated tympanic membrane, especially in children.

One of the major deficiencies of the current devices is the potential for the needle to make contact with crucial physiological structures behind the tympanic membrane, injuring the patient. This potential for injury is amplified in young patients. Although an adult patient is likely to comply with a request to stay still while the health care provider injects him with a four-inch needle, a child is likely to ignore such request.

### BRIEF SUMMARY OF THE INVENTION

This invention addresses the problem of delivering medicine to the middle and inner ear and evacuation of fluid, if any, located in the tympanic cavity. This device overcomes the deficiencies of prior devices by eliminating the need for penetrating into the tympanic cavity beyond the normal physiological position of the tympanic membrane. By doing so, this device presents a novel and safe way for delivering medicine into the tympanic cavity. Instead of penetrating the tympanic membrane by plunging a needle through the membrane and into the tympanic cavity, the present invention flexes the membrane toward a piercing element (or multiple piercing elements). Because the piercing element does not enter the tympanic cavity beyond the normal physiological position of the tympanic membrane, there is no risk of contact with any of the structures in the tympanic cavity. Further, multiple piercing elements can be used. For example, one hollow piercing element can be used to inject the medicine into the tympanic cavity and another to evacuate the fluid from the tympanic cavity. Alternatively, a double-walled piercing element (e.g., a double-lumen needle) can also be used. Also, a hollow piercing element can be used to obtain a biopsy of the tympanic membrane tissue, which can later be analyzed. After the evacuation of fluid, injection of medicine, and/or biopsy is completed, the tympanic membrane is released and will return to its normal physiological position, and the device is removed from the patient's car canal.

### BRIEF DESCRIPTION OF THE DRAWINGS

FIG. 1 shows the side view of a device in accordance with one implementation of the present invention.

FIG. 2 shows the front view of the device of FIG. 1.

FIG. 3 shows the cross-section A-A view of the device of FIG. 1.

FIG. 4 shows the cross-section B-B view of the front body of the device of FIG. 1.

FIG. 5 shows removable capsules in accordance with one implementation of the present invention.

FIG. 6 shows the front view of a rear body in accordance with one implementation of the present invention.

FIG. 7 shows the cross-section C-C view of the device of FIG. 1.

FIG. 8 shows the top view of the front body of the device of FIG. 1 and the top view of the removable housing of FIG. 1.

FIG. 9 shows the side view, top view, and cross-section D-D view of the handle of the device of FIG. 1.

FIG. 10 shows a table with position numbers and names of the corresponding elements.

### DETAILED DESCRIPTION OF THE PREFERRED EMBODIMENTS

Below is the description of the preferred embodiments of this invention. It is recognized, however, that other embodiments would be obvious to those skilled in the art.

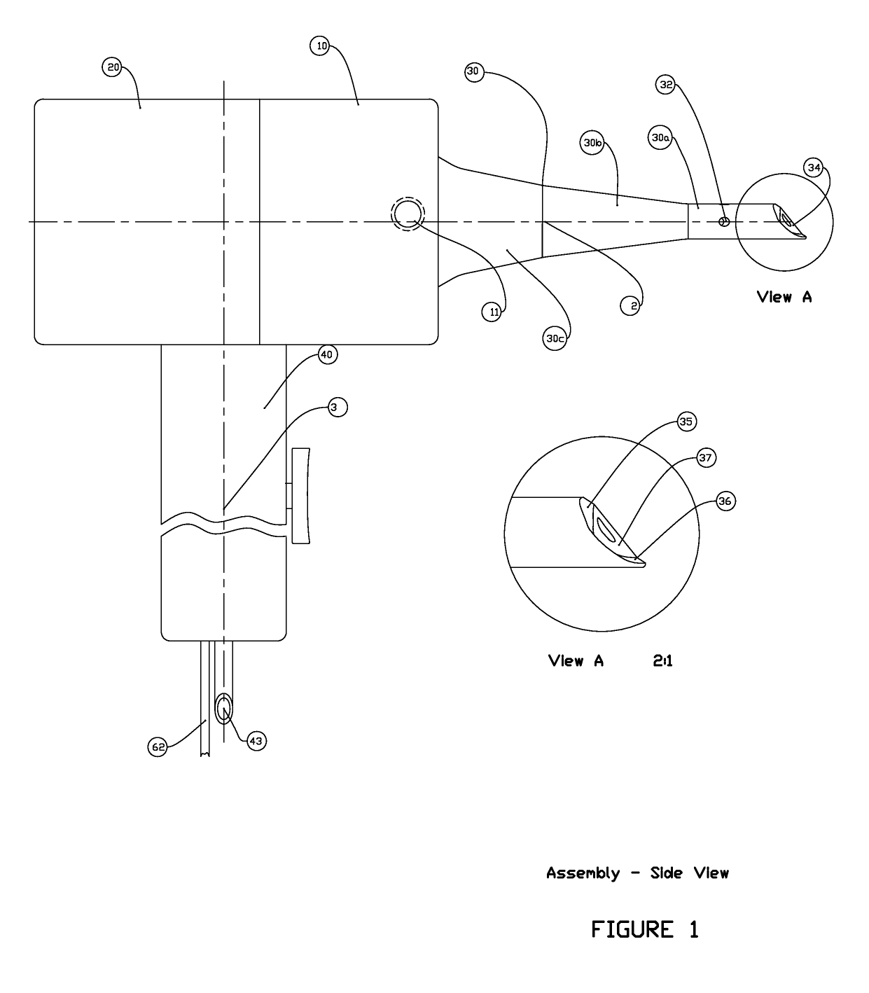

The side view of the device assembly 1 is shown in FIG. 1. In the preferred embodiment, the main components of the device 1 are: front body 10, rear body 20, removable housing 30, and handle 40. In the preferred embodiment, the device creates a slight reduction of pressure in the ear canal, sufficient to flex the tympanic membrane outward, toward the distal surface of the removable housing 34. There are several ways to reduce pressure. In the preferred embodiment, for example, an insufflator bulb, which is commercially available, is connected to the vacuum connection 11. By squeezing the bulb before inserting the device into the car canal, and releasing it after the device has been inserted, the pressure inside the ear canal will be reduced sufficiently enough to flex the tympanic membrane toward the device. There are other means of reducing the pressure in the ear canal exist. For example, a mechanical piston-spring combination can be used, where the piston is preloaded and subsequently released, removing air from the ear canal and creating reduced pressure. Alternatively, a preloaded vacuum capsule or an air pump can be connected to or incorporated in the device to create the same effect. Additionally, there are many alternative ways of reducing the pressure in the ear canal, which are known to those skilled in the art.

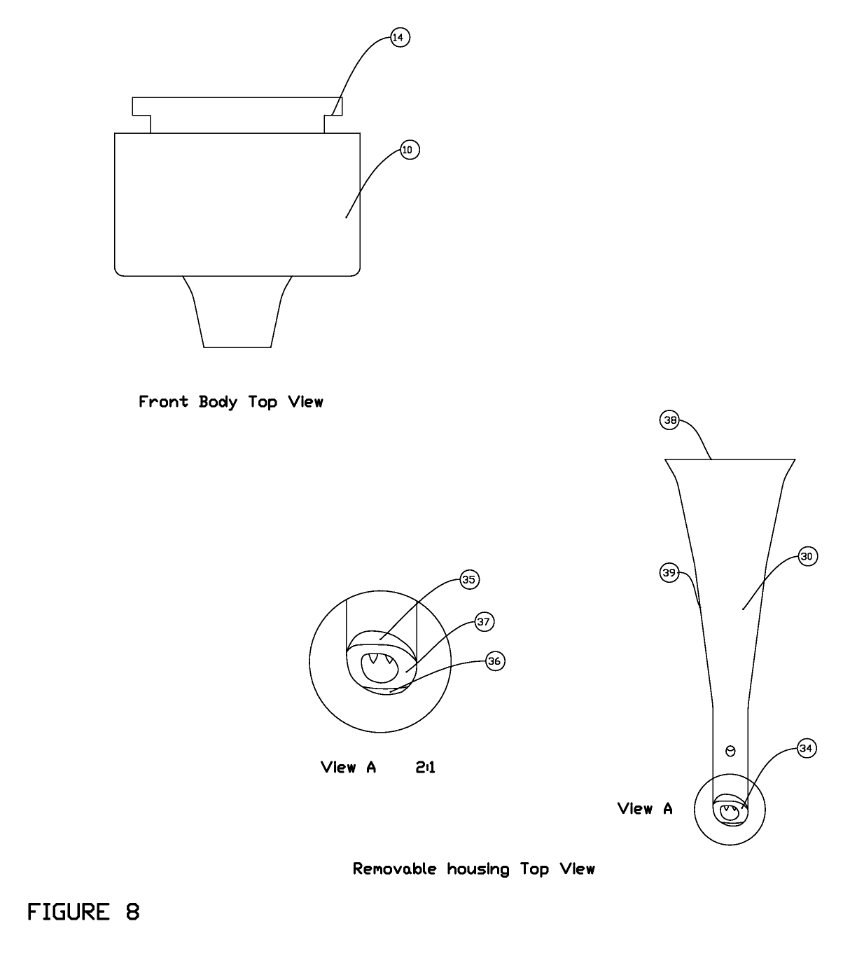

To reduce the pressure in the ear canal, close to the tympanic membrane, it is not necessary to have a perfect seal between the removable housing 30 and the ear canal. Air flow around (and/or across) the tympanic membrane can create a low-pressure zone, and the membrane would subsequently move outward, toward the piercing element(s). However, when there is an airtight seal between the ear canal, the outer ear, and/or the tympanic membrane and the removable housing, the pressure in the ear canal can be reduced quicker and with less airflow in the ear canal. In the preferred embodiment, the removable housing 30 forms an airtight seal with the ear canal. In the preferred embodiment, the outside shape of the removable housing 30 is formed by the distal surface of the removable housing 34, proximal surface of the removable housing 38, and the circumscribed surface of the removable housing 39, shown in FIG. 8, which connects the distal surface and the proximal surface. Although in the preferred embodiment the removable housing 34 is hollow and has a definite wall thickness, it is recognized that the housing may also be solid. To seal the ear canal, the middle section of the housing 30b is made of relatively soft and flexible material, for example silicone or neoprene, which can be squeezed and conformed to the ear canal when the device is inserted. The middle section 30b can be manufactured in many ways known to those skilled in the art. For example, the middle section can be molded in a two-shot molding process, where either front section 30a and back section 30c are molded first, and then middle section 30b is overmolded, or the reverse. It is also recognized that the entire removable housing 30 can be made of or overmolded with relatively soft and flexible material, such a silicone or neoprene. Further, the removable housing can be made of various types of materials. Consequently, the manufacturing process of forming a section that would create a seal depends on the type of material selected for the remaining sections of the removable housing. In the preferred embodiment, other sections of the removable housing (i.e., the front section 30a and back section 30c) can be made from essentially any stable and sufficiently rigid material; for example, plastic suitable for medical use, such as medical grade polypropylene.

A seal may also be formed between the removable housing and the outside ear, or the distal surface of the housing 34 and the tympanic membrane. Also, a soft material is not necessary to form the seal between the housing and the ear canal; such seal can be formed between an inflexible circumscribed surface of the removable housing 39 and the soft tissue of the ear canal.

It is recognized that there are other ways of flexing the tympanic membrane outward (i.e., toward the distal surface of the housing 34). For example, a probe can be incorporated into the housing. After the device is inserted into the patient's ear, the probe would make contact with the tympanic membrane, then attach its tip to the membrane and move outward (i.e., toward the rear body 20), flexing the tympanic membrane outward, toward the distal surface of the housing 34. To attach its tip to the tympanic membrane, for example, the probe can have an adhesive or barbed tip.

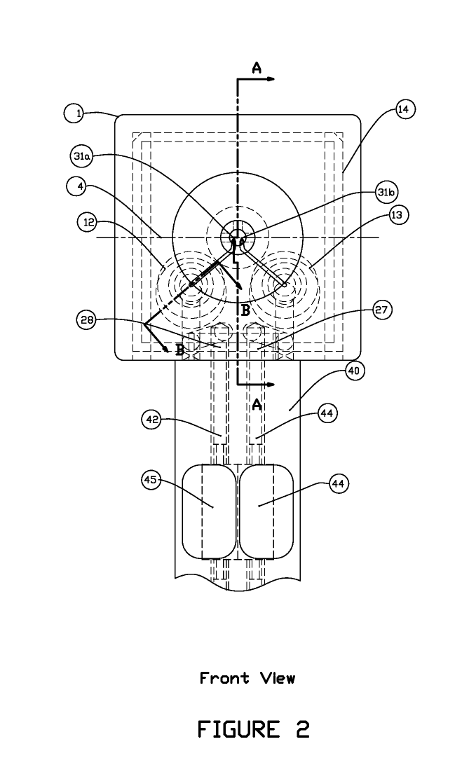

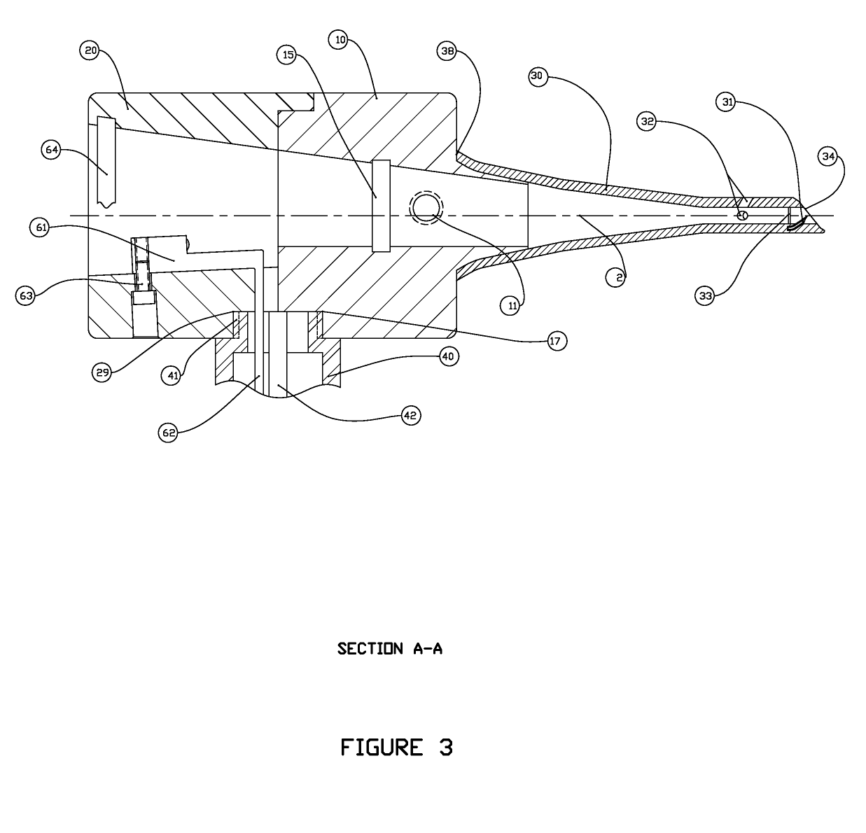

FIG. 3 shows the cross-section A-A. Location of this cross-section is shown in FIG. 2. This figure shows the preferred embodiment of the assembled device 1 and its major components: front body 10, rear body 20, removable housing 30, and handle 40. The vacuum connection 11 can also be seen in this figure. Although the entire device and/or any portion thereof can be made for repeated uses (i.e., non-disposable), in the preferred embodiment, the removable housing 30 is intended to be disposable and should be discarded after a single use.

In the preferred embodiment, removable housing 30 is detachable from the front body 10. Essentially the same device can be made in one piece (i.e., as a uni-body) or comprised of several connected elements. A single-unit device, however, would have to be disinfected or discarded after each use. By contrast, having a disposable removable housing 30 eliminates the need for sterilization of the device after each use, and only a small part of the device is discarded. In one embodiment, the proximal surface of the removable housing 38 can be slightly adhesive, enough to keep the removable housing attached to the front body 10 for the duration of the procedure. For example, a 3M™ Transfer Adhesive 152 can be used. In the preferred embodiment, the front body 10 and the removable housing 30 can have interlocking tapers having a locking angle, for example 8° included angle. In yet another embodiment, the removable housing 30 can snap into the front body 10 (or reverse). In yet another embodiment a twist-lock setup can be implemented, similar to the one described in U.S. Pat. No. 3,749,438. Furthermore, the removable housing 30 can be affixed to the front body 10 in many different ways known to those skilled in the art.

In the preferred embodiment, the vacuum connection 11 is located in the front body 10. Consequently, the connection between the removable housing 30 and the front body 10 has to be sufficiently airtight. There are many ways known to those skilled in the art how this can be accomplished. In the preferred embodiment, the locking taper serves as an air seal. Because the air pressure differential is very small, the seal does not need any additional gaskets.

The piercing element 31a is disposed in the removable housing 30. Position number 31 is used to refer to any piercing element at any position, generally. Positions 31a and 31b specifically refer to piercing elements connected to medicine chamber 12 and evacuation chamber 13 respectively. The device can accommodate one or multiple piercing elements. As discussed above, the tympanic membrane is flexed toward the distal surface of the removable housing 34 when pressure in the ear canal is reduced. The tympanic membrane then self-penetrates when it flexes past the piercing elements 31, shown in FIG. 3. Subsequently, medicine can be injected into and/or fluid can be drawn out of the tympanic cavity. Also, a hollow piercing element can be used to obtain a biopsy of the tympanic membrane.

In the preferred embodiment, the piercing elements 31 are recessed from the distal surface 34—i.e., the tips of the piercing elements 31 do not protrude past the distal surface 34. However, even though recessing the piercing elements makes the device safer, it is not necessary for the basic operation of the device, and the tips of the piercing elements 31 may protrude past the distal surface 34.

In the preferred embodiment the device has two piercing elements 31a and 31b, as shown in FIG. 2. Further, in the preferred embodiment, the piercing elements are hollow. This allows medicine to be delivered through the piercing element into the tympanic cavity. This also allows fluid to be removed from the tympanic cavity, through the same piercing element that is used for delivery of medicine or through another piercing element. The two hollow piercing elements 31 can be used simultaneously or sequentially. Using two piercing elements allows the user to flush the tympanic cavity—flushing out the fluid inside the cavity while injecting medicine. A hollow piercing element can be used to collect a biopsy of the tympanic membrane.

Also, a double-walled piercing element can be used, for example, a double-lumen hypodermic needle. One double-walled piercing element can serve essentially the same function as two hollow piercing elements: it can be used to inject medicine and evacuate fluid from the tympanic cavity, either simultaneously or sequentially, and/or obtain a biopsy of the tympanic membrane.

A solid piercing element can also be used to penetrate the tympanic membrane. Such piercing element can be used, for example, to relieve the pressure inside the tympanic cavity created by the fluid buildup. By making a small perforation in the tympanic membrane, some of the fluid can be drained out, and the pressure inside the tympanic cavity can be equalized with the outside. However, one of the drawbacks to using solid piercing element(s) is that medicine cannot be delivered into and fluid drawn out of the tympanic cavity.

In the preferred embodiment, the piercing element(s) 31 is/are overmolded in the removable housing 30. However, there are also other methods of manufacturing the same, which are known to those skilled in the art. For example, the piercing element 31 can be inserted into a cored-out cavity in the housing, and affixed with glue, ultrasonic welding, or other known methods.

There is a wide variety of medicines that can be delivered into the tympanic cavity. For example, amoxicillin or ciprofloxacin-dexamethasone can be delivered into the tympanic cavity to treat acute otitis media. Also, pain relief medicine can be delivered into the cavity. The present invention can also be used to deliver drugs for inner ear therapies. These drugs can be delivered into the tympanic cavity and can then diffuse into the inner ear from the tympanic cavity.

In the preferred embodiment, the distal surface of the housing 34, shown in FIG. 1, is slightly adhesive. For example, a 3M™ Transfer Adhesive 152 can be used. This helps maintain the tympanic membrane in the flexed-outward position, for example when there is a decrease in vacuum in the ear canal (i.e., increase in pressure). However, it is not necessary for the distal surface of the housing 34 to be adhesive. The tympanic membrane can also remain in the flexed-outward position by maintaining a reduced pressure in the zone immediately near the membrane or in the ear canal. Likewise, the membrane can be held in the flexed-outward position by the piercing elements. However, the piercing elements would have to have appropriate surface roughness and diameter (or barbs) to create sufficient force to prevent the membrane from flexing back into its normal physiological position.

After the procedure is completed, the tympanic membrane is moved away from the piercing element(s), back to its normal physiological position and the device is removed from the ear canal. To aid in moving the tympanic membrane off the piercing elements 31 and back to its normal physiological position, away from the distal surface of the removable housing 34, shown in FIG. 1, the membrane can be pushed inward. This can be done, for example, by slightly increasing the pressure in the car canal—this increased pressure will push the tympanic membrane away from the distal surface of the removable housing 34. In the preferred embodiment, the pressure is increased by squeezing the insufflator bulb, which is connected to the vacuum connection 11. Alternatively, the device can be pulled out without first moving the tympanic membrane away from the distal surface of the removable housing 34 or removing it from the piercing elements. However, moving the tympanic membrane to its normal physiological position reduces the possibility of damaging the membrane when the device is removed from the ear canal. Likewise, there are other ways of moving the tympanic membrane away from the distal surface of the removable housing 34 (e.g., a push probe), which are known to those skilled in the art.

As shown in FIG. 3, in the preferred embodiment, the distal surface of the housing 34 is approximately disposed in a plane oriented at a compound angle with respect to the x-axis of device 2, shown in FIG. 1, such that when the device is inserted in the ear, the distal surface of the housing 34 can be aligned to be approximately parallel to the tympanic membrane. That is, the distal surface of the housing 34 is disposed approximately in a plane that is at an angle with respect to the y-axis of the device 3, shown in FIG. 1, and at an angle with respect to the z-axis of device 4, shown in FIG. 2. It is recognized that the distal surface of the housing 34 can be disposed at various angles with respect to x-axis 2, y-axis 3, and/or z-axis 4. For example, the distal surface of the housing 34 can be approximately disposed in a plane normal to the x-axis 2.

As shown in FIG. 3, in the preferred embodiment, the top portion of the distal surface 35 extends away from the plane of the distal surface of the removable housing 34 slightly more than the bottom portion of the distal surface 36 (for example, 0.4 mm). This offset between the top portion of the distal surface 35 and the bottom portion 36 creates a gap between the bottom portion 36 and the tympanic membrane when the device is inserted into the ear canal and the top portion 35 is in contact with the tympanic membrane. This gap between the bottom portion 36 and the tympanic membrane will be roughly the same as the offset (for example, 0.4 mm). As shown in FIGS. 1 and 8, in the preferred embodiment, the middle portion of the distal surface 37 blends and connects the top portion 35 and the bottom portion 36. However, the distal surface of the removable housing 35 can also be divided into different segments, which can be offset from each other or remain in the same plane. Likewise, the top portion 35 and the bottom portion 36 can also remain in the same plane. Furthermore, the surface of the ear drum is not perfectly flat, and among various shapes that the distal surface of the housing 34 can be made, it can also be made to approximate the shape of the tympanic membrane.

In one embodiment, the middle section of housing 30b, shown in FIG. 1, has an oval-shaped cross-section, which is approximate to the shape of the cross-section of the ear canal at the inflection point, at the beginning of the bony part of the external car canal. This would ensure that the device can be inserted into the ear canal only in one way, and would also ensure that the distal surface of the device and the piercing elements are in the right location. Consequently, in the preferred embodiment, the distal surface, as shown in FIG. 3 and described above, is approximately parallel to the tympanic membrane. Further, the piercing elements 31a and 31b line up such that when the tympanic membrane is flexed outward they will pierce it in the posteroinferior and/or anteroinferior quadrants. However, the piercing elements can be located in a way to pierce any and all of the quadrants of the tympanic membrane.

As shown in FIG. 3, in the preferred embodiment, the removable housing 30 has a fluid-blocking wall 33. Because in the preferred embodiment the removable housing is disposable and the remaining components of the device are not, it is preferable that all bodily fluids are kept away from the non-disposable parts to eliminate the need for sterilization of those parts. The fluid-blocking wall 33 prevents any fluid, which may escape around the piercing elements, from contaminating the internal non-disposable elements of the device.

In the preferred embodiment, to reduce the pressure in the ear canal, the air from the canal is drawn through vacuum ports 32, which are disposed in the removable housing 30. Consequently, it is not necessary for the fluid-blocking wall 33 to be gas-permeable. However, if the device will be used in conjunction with an otoscope, the fluid-blocking wall must be transparent, so that the user is able to view the tympanic membrane through the removable housing. There are many materials, which are known to those skilled in the art that can be used to accommodate this. In the preferred embodiment, the fluid-blocking wall 33 is made from either clear acrylic or polycarbonate. The fluid-blocking wall 33 can be affixed to the removable housing in a variety of ways known to those skilled in the art. For example, the fluid-blocking wall can be ultrasonically welded to the removable housing 30 or overmolded. On the other hand, if the device is not used in conjunction with the otoscope, and the user does not need to be able to view the tympanic membrane, the fluid-blocking wall can be made from any material that would be best incorporated in the housing. There are many different ways known to those skilled in the art in which a fluid-blocking wall 33 can be constructed. Also, there are many materials that are known to those skilled in the art, which can be used to construct the fluid-blocking wall 33. Furthermore, the fluid-blocking wall is not necessary for the basic function of the device.

In one embodiment, the vacuum ports 32 are sealed with an air-permeable but fluid-blocking membrane, for example a membrane made from a porous plastic. This would allow for removal of air from the car canal but would prevent fluid from passing through the vacuum ports. In another embodiment, the entire removable housing or any portion thereof (for example the front section 30a) can be made from porous plastic, which would allow the air to flow through the walls of the removable housing but would prevent fluid from contaminating the non-disposable parts of the device. Using porous plastic in the removable housing can also eliminate the need for vacuum ports.

In one embodiment, the pressure in the ear canal is reduced by removing the air from the ear canal through one of the piercing elements. The same element that is used for delivery, evacuation, or biopsy can be used as a vacuum port for pressure reduction. A double-walled piercing element (e.g., double-lumen needle) can also be used in the same manner, wherein one of the flow channels is used to remove air from the ear canal, reducing the pressure therein.

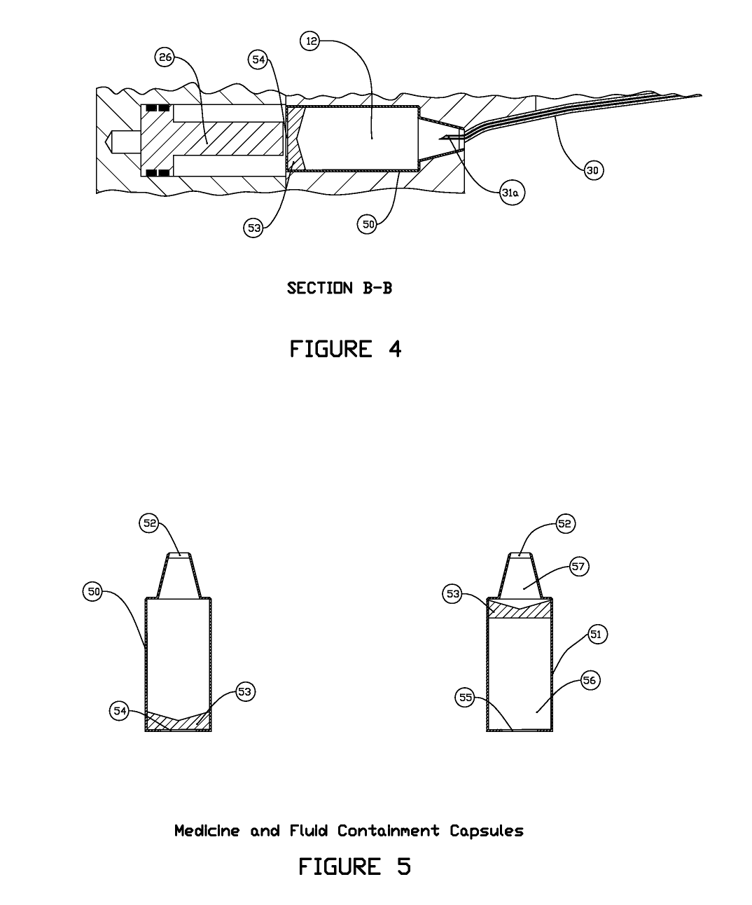

In the preferred embodiment, shown in FIG. 4, the piercing elements 31 are connected to different chambers. Piercing element 31a is connected to the medicine capsule 50, located in the medicine chamber 12. This connection is accomplished by piercing the rubber seal 52, shown in FIG. 5, on the medicine capsule 50 with the proximal end of the piercing element 31a. Subsequently, after the tympanic membrane is pierced by the piercing element 31a, the medicine located in capsule 50 can flow through the piercing element and into the tympanic cavity. Piercing element 31b is connected to the containment capsule 51, located in the evacuation chamber 13. This connection is accomplished by piercing the rubber seal 52, shown in FIG. 5, on the fluid containment capsule 51, with the proximal end of the piercing element 31b. Subsequently, after the tympanic membrane is pierced by the piercing element 31a, any fluid located in the tympanic cavity can flow through the piercing element into the fluid containment capsule 51. There are many ways known to those skilled in the art, for making a functionally-equivalent connection between a piercing element and a capsule.

As shown in FIG. 4, in the preferred embodiment, the medicine capsule 50 is inserted into the medicine chamber 12, which is disposed in the front body 10. Likewise, in the preferred embodiment, the fluid evacuation capsule 51, shown in FIG. 5, is inserted into the evacuation chamber 13, which is disposed in the front body 10, shown in FIG. 2. In the preferred embodiment, both the medicine capsule 50 and the fluid evacuation capsule 51 are disposable and are designed to be discarded after single use (although the evacuated fluid can be used for research and analysis of the otopathogen). It is recognized, however, that there are many embodiments of the device in which these capsules would not be located in the front body 10—for example, the capsules 50 and 51 and/or chambers 12 and 13 can be integrated into the removable housing 30. Alternatively, the entire front body 10 can also be made disposable or sterilized after each use, to eliminate the need for capsules altogether and use the chambers 12 and 13 without capsules.

There are many materials known to those skilled in the art, which can be used for the medicine and fluid containment capsules. For example, medical grade polypropylene can be used for the body of the capsule and neoprene can be used for the rubber seal.

As shown in FIG. 3, in the preferred embodiment, there is a sealing lens 15 disposed in the central opening 16 of the front body 10. The purpose of the sealing lens 15 is to reduce the total volume of air that needs to be removed to create sufficient pressure reduction in the ear canal. The sealing lens 15 can also be a magnifying lens, used to improve visibility of the tympanic membrane. However, the scaling lens 15 is not necessary for creating a pressure reduction in the ear canal.

As shown in FIG. 3, in the preferred embodiment, the front body 10 can be made of essentially any sufficiently rigid and stable material. For example, the front body 10 can be made of plastic, aluminum, stainless steel, etc. In the preferred embodiment, the sealing lens 15 is made of clear plastic, for example acrylic or polycarbonate. The lens can then be snapped into the groove in the central opening 16 in the front body. Further, it is not necessary to have a perfectly airtight seal between the front body 10 and the sealing lens 15 for the device to function properly. However, an airtight seal would improve the functionality of the device. The seal can be created, for example, by placing a rubber gasket either around the circumference of the lens or in the groove, or sealing the lens in place with silicone. It is recognized that there are many ways that are known to those skilled in the art in which the same can be accomplished. Further, it is also recognized that the sealing lens 15 does not have to be made from transparent material if the user does not intend to view the tympanic membrane of the patient. Although the sealing lens improves functionality of the device, it is not essential for its operation.

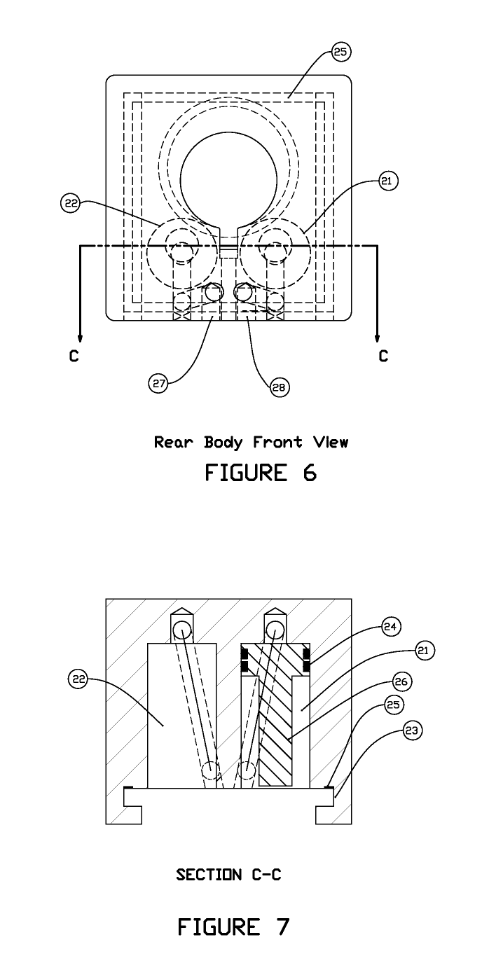

The rear body 20 is shown in FIGS. 6 and 7. In the preferred embodiment, the front body 10 is connected to the rear body 20 by sliding the T-slot connection 14 into the T-slot 23. After the front body 10 and the rear body 20 are connected, the threaded end 41 of the handle 40 is screwed into the threaded hole formed by threaded half-hole 17 and threaded half-hole 29. This ensures that the front body 10 and the rear body 20 do not slide apart. There are many other ways in which this portion of the device can be made, which are known to those skilled in the art. It is further recognized that these connections are not central to the invention.

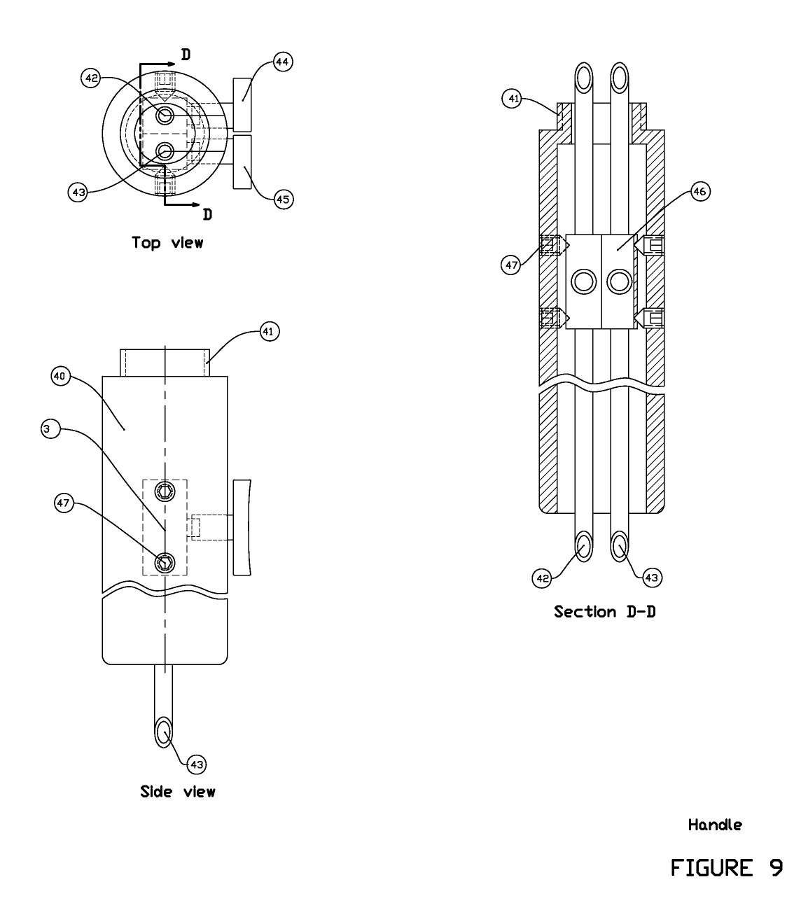

In the preferred embodiment, the evacuation mechanism 22 is pneumatically activated. The vacuum line 43, is connected to the internal vacuum lines 27, shown in FIG. 2. The air valve 46, shown in FIG. 9, restricts airflow through the line 43 until evacuation trigger 45 is pressed. When the evacuation trigger 45 is pressed, the air starts to flow through the air line 43, and out of the internal vacuum lines 27. Then, the air flows out of the chamber of the evacuation mechanism 22, shown in FIG. 7. Subsequently, the air outflow creates a pressure differential between the reserved space 57 and fluid space 56 of the fluid containment capsule 51, shown in FIG. 5 The higher pressure in the reserved space 57 pushes the plunger 53 toward the back of the fluid containment capsule 52 (i.e., into the fluid space). Subsequently, this creates air and/or fluid flow from the tympanic cavity, through the piercing element 31b into the fluid containment capsule 51.

In the preferred embodiment, the injection mechanism 21 is pneumatically activated. The air line 42, is connected to the internal air lines 28, shown in FIG. 2. The air valve 46, shown in FIG. 9, restricts airflow through the line 42 until medicine trigger 44 is pressed. When the medicine trigger 44 is pressed, the air starts to flow through the air line 42 and into the internal air lines 28. Subsequently, the air pushes forward piston 26, shown in FIG. 7, of the injection mechanism 21; piston seals 24 prevent the air from flowing around the back portion of the piston. The piston then makes contact with the plunger 53, shown in FIGS. 4 and 5, of the medicine capsule 50. The plunger then pushes forward the medicine contained in the medicine capsule 50, which flows through the piercing element 31a.

In the preferred embodiment, the front gasket 25, shown in FIG. 7, prevents air leaks between the front body 10 and the rear body 20, and helps to facilitate the proper function of the injection mechanism 21 and evacuation mechanism 22.

As shown in FIGS. 6 and 7, in the preferred embodiment, the injection mechanism 21 and evacuation mechanism 22 are located in the rear body 20. However, the location of these mechanisms is not central to this invention. There are various ways for injecting medicine into and evacuating the fluid out of the tympanic cavity, and these mechanisms can be located in any part of the device. In the preferred embodiment, air pressure is used to actuate both mechanisms. This is not central to the invention, and the same functions can be performed by mechanical and/or electro-mechanical mechanisms.

In the preferred embodiment, an otoscope is incorporated into the rear body 20 to allow the user to view the tympanic membrane. The LED light 61, shown in FIG. 3, is used to illuminate the ear canal and the tympanic membrane. The LED light 61 is connected to a power source via power cable 62 and is fastened to the rear body 20 by the socket head cap screw 63. A rear lens 64 seals and protects the components inside the rear body 20. In the preferred embodiment, the rear lens 64 also serves to magnify the view. It is not necessary, however, for the device to have the viewing capabilities, and the device can be used by a user without viewing the ear canal and/or the tympanic membrane.

In some instances, the tympanic membrane might be bulging from the fluid buildup in the tympanic cavity. In some of these instances it may be unnecessary to flex the tympanic membrane any more outward than it already has been flexed from its normal physiological position by the fluid buildup. However, the same device can still be used to penetrate the tympanic membrane in a safe manner. In the preferred embodiment of the device the piercing elements 31 are recessed from the distal surface of the removable housing 34. Consequently, when the distal surface 34 contacts the bulging tympanic membrane, the membrane would sink into the recess and would be penetrated by the piercing element(s). Because the elements are recessed from the distal surface, it/they present no danger to any of the internal structures inside the tympanic cavity.

To help with using the device for treatment of children, an acoustic device (e.g., a speaker) can be incorporated in the device, to entertain or distract the patient before and/or during the procedure. For example, a small speaker can be incorporated into the front body 10, which can produce various distracting sounds, such as music, which would divert the child's attention away from the procedure.

It is to be appreciated that there are many alterations and modifications of this invention, which will be apparent to those skilled in the art, that are intended to be within the spirit and scope of this invention.

1-34. (canceled)

35. A device for substance delivery to and/or extraction from a patient's tympanic cavity, the device comprising:
a housing having a distal surface, a proximal surface that is opposite to the distal surface, and a circumscribed surface that connects the distal surface and the proximal surface;
at least one piercing element disposed within the housing, and the at least one piercing element having at least one proximal end and at least one distal piercing end,
wherein the at least one distal piercing end is configured to pierce the patient's tympanic membrane and is generally facing the distal surface of the housing; and
a means for flexing the patient's tympanic membrane toward the at least one distal piercing end of the at least one piercing element.

36. The device as recited in claim 35, wherein the at least one piercing element comprises at least one hollow piercing element.

37. The device as recited in claim 36, further comprising at least one chamber in fluid communication with the at least one hollow piercing element.

38. The device as recited in claim 37, further comprising at least one capsule located in the at least one chamber.

39. The device as recited in claim 35, wherein the at least one piercing element is recessed from the distal surface of the housing.

40. The device as recited in claim 35, wherein a cross-section of the circumscribed surface that connects the distal surface and the proximal surface is of approximately the same shape as a cross-section of the patient's ear canal at a point of inflection.

41. The device as recited in claim 35, wherein the distal surface of the housing is slightly adhesive.

42. The device as recited in claim 35, wherein the distal surface of the housing is disposed at a compound angle with respect to a longitudinal axis of the housing.

43. The device as recited in claim 35, further comprising one or more triggers for activating one or more of evacuation of substance from the patient's tympanic cavity and injection of substance into the patient's tympanic cavity.

44. A device for substance delivery to and/or extraction from a patient's tympanic cavity, the device comprising:
a housing configured to fit into the patient's ear canal;
at least one piercing element disposed within the housing, the at least one piercing element being sized and configured to pierce the patient's tympanic membrane and is generally oriented to face the patient's tympanic membrane; and
a flexing mechanism configured to flex the patient's tympanic membrane toward the at least one distal piercing element.

45. The device as recited in claim 44, wherein:
the flexing mechanism is configured to create a pressure differential between the patient's tympanic cavity and the patient's ear canal; and
the pressure differential is sufficient to flex the patient's tympanic membrane outward and to penetrate the patient's tympanic membrane on the at least one piercing element.

46. The device as recited in claim 45, further comprising one or more chambers in fluid communication with the at least one piercing element.

47. A method of penetrating a patient's tympanic membrane, the method comprising:
inserting a device into the patient's ear canal, wherein the device comprises at least one piercing element;
flexing the patient's tympanic membrane toward the at least one piercing element of the device; and
penetrating the patient's tympanic membrane with the at least one piercing element.

48. The method as recited in claim 47, wherein:
the at least one piercing element comprises at least one hollow piercing element; and
the method further comprises suctioning a substance out of the patient's tympanic cavity, through the at least one hollow piercing element.

49. The method as recited in claim 47, wherein:
the at least one piercing element comprises at least one hollow piercing element; and
the method further comprises delivering medication into the patient's tympanic cavity, through the at least one hollow piercing element.

50. The method as recited in claim 47, wherein flexing of the patient's tympanic membrane toward the at least one piercing element comprises reducing pressure in the patient's ear canal below atmospheric pressure.

51. The method as recited in claim 47, wherein flexing the patient's tympanic membrane toward the at least one piercing element forces the patient's tympanic membrane to penetrate when the patient's tympanic membrane flexes past the at least one piercing element.

52. The method as recited in claim 47, wherein:
the at least one piercing element comprises a plurality of hollow piercing elements;
the method further comprises simultaneously suctioning a substance out of the patient's tympanic cavity through at least one hollow piercing element of the plurality of piercing elements and injecting medication into the patient's tympanic cavity through at least one hollow piercing element of the plurality of hollow piercing elements.

53. The method as recited in claim 47, wherein:
the at least one piercing element comprises a plurality of hollow piercing elements; and
the method further comprises flushing the patient's tympanic cavity.

54. The method as recited in claim 47, further comprising forming a seal between the device and the patient's ear canal.
